(19) United States
(12) Patent Application Publication
(10) Pub. No.: US 2006/0213513 A1
(43) Pub. Date: Sep. 28, 2006

(54) Portable breathing apparatus

(76) Inventor: Sae-Jae Seong, Seoul (KR)

Correspondence Address: DILWORTH & BARRESE, LLP, 333 EARLE OVINGTON BLVD., UNIONDALE, NY, 11553, US

(21) Appl. No.: 11/088,593

(22) Filed: Mar. 24, 2005

(51) Int. Cl. A61M 15/00; A61M 16/10

(52) U.S. Cl. 128/203.12

(57) ABSTRACT

The portable breathing apparatus according to the present invention includes an air tank chargeable with air at the predetermined pressure and portable, a pressure valve member bypassing one portion of air when the pressure of air supplied from the air tank falls less than certain pressure, a regulator valve member converting air induced from the pressure valve member into breathable pressure, thereby regularly supplying the converted air to a mouth, a pressure switching member enabling electric power to be switched on/off by compressed air, induced and bypassed from the pressure valve member, and a vibration means, contactable to a human body, generating vibration at the same time when the electric power of the pressure switching member is switched on, thereby warning lack of compressed air. Thus, the present invention may reduce casualties by timely informing an operator of the time when the carrying air tank is to be supplemented while operating at a crisis and very hectic site such as diving or the fire, and at the same if necessary, to enable the operator to ask for help to another person nearby.

### BACKGROUND OF THE INVENTION

1. Field of the Invention

The present invention relates to a portable breathing apparatus, more particularly, to a portable breathing apparatus informing the lack of air automatically or manually when the pressure of air supplied from an air tank falls down less than an appropriate level, or when a user wants to gives a notice of one's danger

2. Description of the Related Art

Generally, portable breathing apparatus are safety equipments enabling divers under water or fire fighters at the fire to carry the portable breathing apparatus therewith and safely breathe at the place where normal breathing is impossible.

The conventional portable breathing apparatus comprises, generally, a portable air tank 10, a pressure valve member 20, a regulator valve member 30 and an alarm generator 40. That is, the portable air tank 10 is provided to be carriable on the back using a general portable means 11, to be charged with air at the pressure of about 200˜300 kg/cm³ inside thereof 10 , and to be lasting about 50 minutes with this amount of air.

Moreover, the pressure valve member 20, a three way valve, enables air more than about 60 kg/cm³, on one side, to be supplied into a mouth 31, thereby making breathing possible and, when the pressure of air bypassing one side like above falls down less than 60 kg/cm³, enables an alarm sound to be generated by the alarm generator 40 mounted at one end of the bypass pipe 21 while one portion of air is being bypassed.

Namely, the extremely high pressure of compressed air is transmitted to the pressure valve member 20 from the portable air tank 10, and when the pressure of air exceeds 60 kg/cm³, the bypass pipe 21 is closed, the whole quantity of air is transmitted to the regulator valve member 30, the transmitted air is converted into the state of breathable pressure and transmitted to the mouth 31. Thus, safe breathing is possible.

When the pressure of air supplied from the air tank 10 falls down less than 60kg/cm³ while safe breathing through normal air supply like above, the pressure valve member 20 lets air flow not only into the side of the regulator valve member 30, but into the side of the bypass pipe 21, thereby informing the lack of compressed air.

The alarm means like above is for informing recharge of air to the air tank 10, by letting adjacent people as well as an operator oneself recognize the lack of air at the same time, because it is impossible to know when air of the air tank 10 is short.

However, an alarm using a sound like above has week effectiveness under water or at the fire site.

Because an alarm sound is inaudible due to noise of surroundings and tension, even the operator oneself may not predict amount of compressed air and worsened situation in the state of pressure. In addition, in case that there are several persons together at the same time, alarm sounds arouse here and there, thus, it is impossible to sense whose alarm sound is alarming, and when diving, it is very inconvenient that a diver has to check one's own residual pressure through a residual pressure gauge.

Especially, in case that the operator does not hear the alarm sound at the urgent site or just misses the time to check the residual pressure through the residual pressure gauge, it might cause a serious problem such as casualties.

### SUMMARY OF THE INVENTION

The present invention is conceived to solve the aforementioned problems in the prior art. The present invention provides a breathing apparatus capable of informing an exact alarm at the site where it is very noisy or where a visual field is bad, by enabling a user oneself to sense the event sharply when the pressure of an air tank falls down less than a standard level.

In order to achieve the above object, the present invention comprises: an air tank chargeable with air at the predetermined pressure and portable; a pressure valve member bypassing one portion of air when the pressure of air supplied from the air tank falls less than certain pressure; a regulator valve member converting air induced from the pressure valve member into breathable pressure, thereby regularly supplying the converted air to a mouth; a pressure switching member enabling electric power to be switched on/off by compressed air, induced and bypassed from the pressure valve member; and a vibration means, contactable to a human body, generating vibration at the same time when the electric power of the pressure switching member is switched on, thereby warning lack of compressed air.

### BRIEF DESCRIPTION OF THE DRAWINGS

The above and other features and advantages of the present invention will become more apparent by describing in detail exemplary embodiments thereof with reference to the attached drawings in which:

FIG. 1 is a view showing the schematic structure of the conventional portable breathing apparatus;

FIG. 2 is a block view showing one embodiment of the portable breathing apparatus according to the present invention; and

FIG. 3 is a block view showing the other embodiment of the present invention.

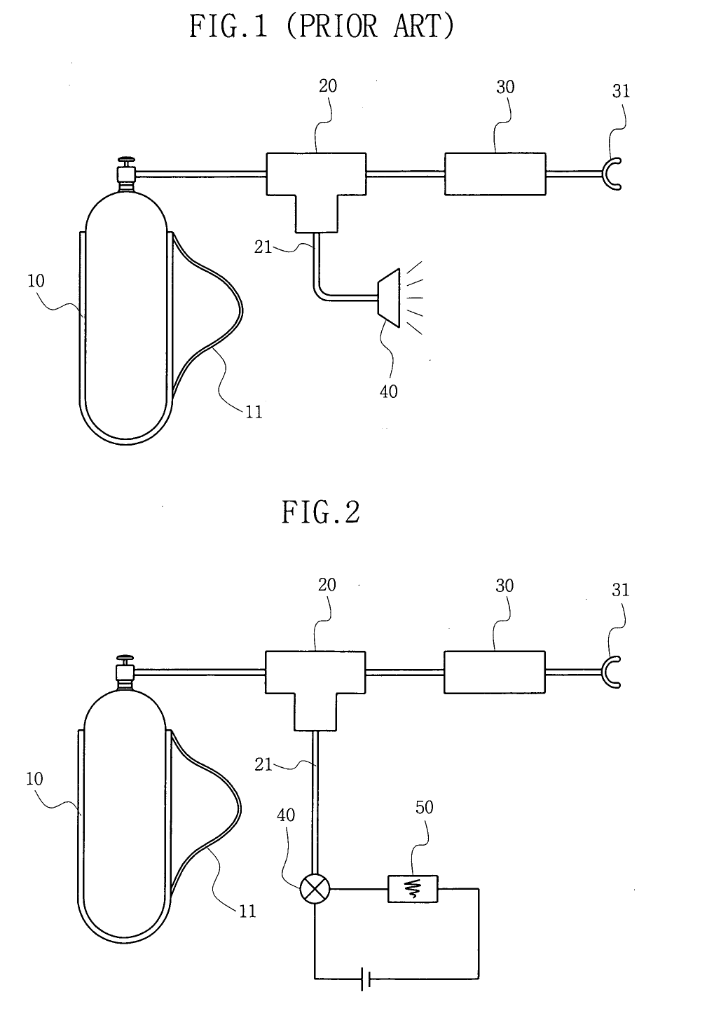

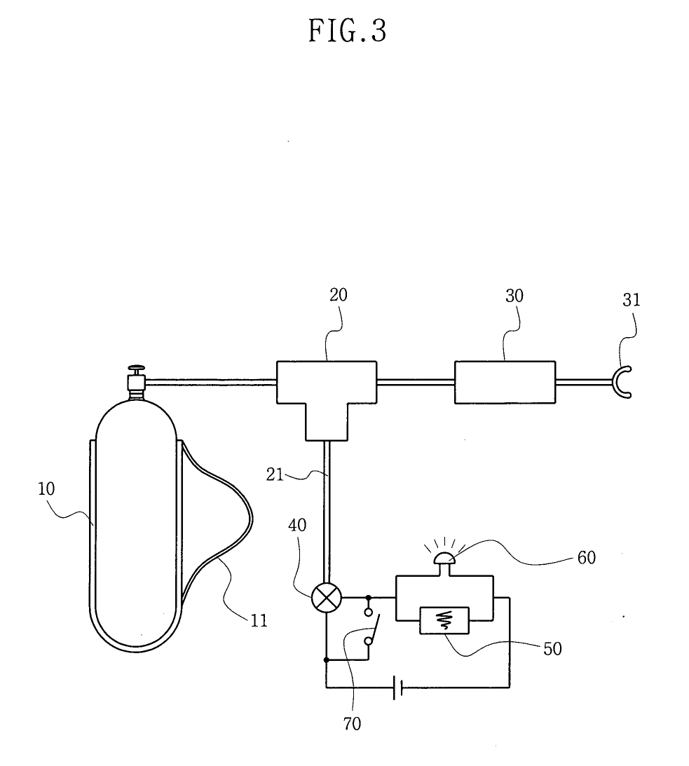

### DETAILED DESCRIPTION OF THE INVENTION

Hereinafter, preferred embodiments of the present invention will be in detail described with reference to the accompanying drawings.

The present invention comprises an air tank 10, a pressure valve member 20, a regulator valve member 30, a pressure switching member 40, and a vibration means 50, as illustrated in FIG. 2.

At this time, the air tank 10, the pressure valve member 20, and the regulator valve member 30 are provided as same as in the previous configuration. Moreover, in the present invention, there is further provided the vibration means 50 together with the pressure switching member 40, which is the most outstanding characteristic of the present invention.

The air tank 10 is provided into a portable means 11 enabling the air tank 10 to be carriable on the back, so as not to be left off, and at the same time, air is compressed at the certain pressure inside of the air tank 10.

Although there is a little difference in the pressure of compressed air in accordance with usage thereof, generally, in case of the air tank 10 for diving, the pressure of air is compressed at about 200 kg/cm³ and in case of the air tank 10 for fire fighters at the fire site, at about 300 kg/cm^{3.}

The pressure valve member 20 is formed of a three way valve, letting compressed air induced from the air tank 10 to pass, and in case that the pressure of compressed air falls down less than a certain level, enabling one portion of air to be bypassed by letting a passage on one side, closed by compressed air, be opened.

The control pressure at the pressure valve member 20 is generally based on 60 kg/cm³ and in case that the pressure of compressed air passing therethrough 20 falls down less than 60 kg/cm³, a closed passage is opened, and thereby, one portion of air is enabled to be bypassed through the bypass pipe 21.

The regulator valve member 30 enables compressed air, induced from the pressure valve member 20, to be converted into the breathable pressure and be supplied to a mouth 31 thereby. At this time, the mouth 31 is provided at a portion where air at the certain pressure through the regulator valve member 30 is outlet and formed so that a user may bite the mouth 31 using one's mouth.

The pressure switching member 40 is provided to switch on/off electric power by the pressure of air induced into the bypass pipe 21, and disconnected at a normal state, however, in case that the pressure of compressed air falls down less than 60 kg/cm³, a passage on one side of the bypass pipe 21 is opened and the electric power is switched on by compressed air induced through the bypass pipe 21.

The vibration means 50 is enabled to operate in accordance with on/off of the pressure switching member 40. That is, when the pressure switching member 40 is switched on by the pressure of compressed air induced into the bypass pipe 21 through the pressure valve member 20, at the same time, the power is approved and the vibration means operates thereby.

Especially, it is preferable that the vibration means 50 is provided to be directly contacted to a human body, such as a shoulder ban or the like, i.e. an air tank portable means. More preferably, the vibration means 50 is provided inner side of the shoulder band or at a portion contacting the back, so that a user may sense the vibration most sharply.

In the meantime, according to the present invention, together with the vibration means 50, at the same time, there may be provided a flashing means 60 to be sensed even by adjacent another person. That is, in case that the flashing means 60 operates at the same time with the vibration means 50, it may let adjacent another person, besides the user oneself, sense the lack of air remaining inside of the air tank 10. Accordingly, in case that the user misses the alarm, the lack of residual air may be recognized by adjacent another person.

Moreover, the vibration means 50 and the flashing means 60 may operate by separate operation of the switch 70. That is, in case that a person carrying the air tank 10 operates the switch 70 to ask for another person's help, the flashing means 60 starts flashing, so that adjacent another person may recognize the flashing immediately and help the user.

Operations of the present invention like above will be described as follows.

The portable breathing apparatus of the present invention is a personal safety equipment, individually portable and being supplied with necessary air during a certain time.

Accordingly, as same as the previous case, in case that a main valve on one side of the air tank 10 is opened in such a manner that a user carries the air tank 10 on the back using the air tank portable means 11 and bites the mouth 31 using one's mouth, the user may perform a necessary operation even at a situation where air is thin.

Namely, in case that air inside of the air tank 10 is consumed during operations under water or at the fire site, amount of air reduces from the air tank 10 and the pressure of air also reduces. Therefore, in case that a certain time elapses, the pressure of air coming from the air tank 10 falls down less than 60 kg/cm^{3.}

In case that the pressure of compressed air falls down less than 60 kg/cm³ the pressure valve member 20 automatically opens a passage on one side of the bypass pipe 21, thereby supplying one portion of compressed air therethrough 21 together with the regulator valve member 30.

In case that compressed air is induced into the bypass pipe 21, the pressure switching member 40 is switched on by the compressed air, and thereby electric power is approved and vibration is generated from the vibration means 50.

Like above, in case that the pressure of residual air at the air tank 10 falls down less than a certain level, the pressure switching member 40 operates by one portion of compressed air, and thereby, the vibration means 50 drives. Thus, an operator oneself carrying the air tank 10 may recognize the lack of air.

Accordingly, the present invention is enabled to prevent an accident by lack of air at the air tank 10 during operations at a site where air is thin.

Meanwhile, in case that residual air at the air tank 10 is short, it is of course, and in case that residual air is enough, however, an operator carrying the air tank 10 is hit by an accident or in a situation needing another person's help, the operator may operate separate switch 70, so as to let the vibration means 50 and the flashing means 60 operate, so that adjacent another person may recognize the event.

Like above, the present invention enables a safer operation and prevents casualties caused by lack of air at the air tank 10, in such a manner that an alarm sound and a replenishment time in accordance with lack of amount of air, i.e. residual pressure at the portable air tank 10 may be exactly recognized.

In addition, in case that the operator needs another person's help in a particular situation, i.e. in a situation where the operator cannot solve difficulty alone, the operator may easily ask for adjacent another person's help.

As aforementioned, the present invention is provided to reduce casualties by timely informing an operator of the time when the carrying air tank 10 is to be supplemented while operating at a crisis and very hectic site such as diving or the fire, and at the same if necessary, to enable the operator to ask for help to another person nearby. Thus, there is an effect that safety in operation is largely improved.

What is claimed is:

1. A portable breathing apparatus comprising:
an air tank chargeable with air at the predetermined pressure and portable;
a pressure valve member bypassing one portion of air when the pressure of air supplied from the air tank falls less than certain pressure;
a regulator valve member converting air induced from the pressure valve member into breathable pressure, thereby regularly supplying the converted air to a mouth;
a pressure switching member enabling electric power to be switched on/off by compressed air, induced and bypassed from the pressure valve member; and
a vibration means, contactable to a human body, generating vibration at the same time when the electric power of the pressure switching member is switched on, thereby warning lack of compressed air.

2. The apparatus of claim 1, wherein the pressure valve member enables one portion of air to be bypassed into a bypass pipe in case that the pressure of passing air reaches less than 60kg/cm^{3.}

3. The apparatus of claim 1, wherein the pressure switching member operates and controls the vibration means, and at the same time, a flashing means letting adjacent another person to know the lack of residual pressure of the air tank.

4. The apparatus of claim 3, wherein the vibration means and the flashing means enable an operator carrying the air tank to operate the same alone by operation of a switch operated by a separate hand operation.
